(19) United States
(12) Patent Application Publication
(10) Pub. No.: US 2012/0301505 A1
(43) Pub. Date: Nov. 29, 2012

(54) Immunostimulating Agent and Method for Production Thereof

(76) Inventors: Kazuya Iwai, Osaka (JP); Takemi Ueda, Osaka (JP); Nanaka Gotoda, Osaka (JP); Keiko Furuya, Osaka (JP); Michihiro Takagi, Hyogo (JP)

(21) Appl. No.: 13/298,639

(22) Filed: Nov. 17, 2011

Related U.S. Application Data
(63) Continuation of application No. 12/281,211, filed on Aug. 29, 2008.

(30) Foreign Application Priority Data
Mar. 1, 2006 (JP) JP 2006-054373

(51) Int. Cl. A61K 31/715; C07H 1/08; A61P 37/04; A61P 37/02

(52) U.S. Cl. 424/278.1; 536/123.1

(57) ABSTRACT

The object is to provide: a safe immunomodulating agent which can be used as a pharmaceutical or a food material; a method for production of the immunomodulating agent; and a novel application of a coffee extract residue. Disclosed is an immunomodulating agent comprising a coffee extract as an active ingredient. Preferably, the coffee extract is an extract containing arabinogalactan. The immunomodulating effect of the immunomodulating agent relies on the promotion of the proliferation of an immunocompetent cell such as a macrophage. The immunocompetent cell is preferably any one selected from a macrophage like strain RAW264 or J774.1, a murine splenocyte, a murine peritoneal macrophage and a murine dendrocyte. A composition containing the immunomodulating agent can be used as a composition such as a pharmaceutical composition, a food composition and a cosmetic composition.

### RELATED APPLICATIONS

To the fullest extent possible, the present application claims priority to, and incorporates by reference, PCT/JP2007/053747 filed Feb. 28, 2007 and JP2006-054373 filed Mar. 1, 2006.

### BACKGROUND OF THE INVENTION

The present invention relates to immunomodulating agent comprising coffee extract as active ingredient. More specifically, the present invention relates to immunomodulating agent comprising arabinogalactan included in coffee extract as active ingredient and its utilization.

### BACKGROUND ART

Conventionally, arabinogalactan extracted from larch has been mainly used. When arabinogalactan from larch (Larch wood AG; L-AG) is used as food additives it is necessary for purifying highly to defecate. A method for production and extraction of arabinogalactan, which can be easily purified from material derived from eatable food, is demanded.

Arabinogalactan from larch is characterized by high water solubility and low viscosity because molecular weight is from about 15000 to 18000. This characteristic enables to add to food as water-soluble fiber without change to texture, and to develop health-conscious food. As it is possible to increase solid content concentration without increasing viscosity, the characteristics of improving transfer and of elevation of pigment stability are given in ink. Transcription of ink can be enhanced in high end ink used on precisely printing of food packing materials, labels, wrapping materials and so on which are necessary for luster and transparence and pigmentary stability can be increased in low end ink used on printing of newspaper, catalog, cardboard and so on.

Thus arabinogalactan from larch is polysaccharide available for a variety of uses.

Meanwhile, coffee beans have high arabinogalactan content. Arabinogalactan from coffee is characterized by larger molecular weight than arabinogalactan from larch. Because of large molecular weight, it can't increase solid content concentration without increasing viscosity. Therefore the uses like arabinogalactan from larch could have not been expected.

Although arabinogalactan is contained in green coffee beans, roasted coffee beans and coffee extract residue, it has not been used as resource of arabinogalactan. So it is demanded a novel application of coffee extract residue, especially fraction contained arabinogalactan.

Meanwhile, it is anticipated the population ages and fewer babies are born from now on and aged men increase, novel pharmaceutical and food material improving immunity are demanded.

Patent literature 1: Japanese Unexamined Patent application Publication No. 2005-8616

### DISCLOSURE OF THE INVENTION

### Problem to be Solved by the Invention

The object of the present invention is to provide a safe immunomodulating agent, which can be used as a pharmaceutical or a food material and the method for production. And a novel application of a coffee extract residue is provided.

### Means for Solving the Problem

To achieve the above objects, devoting themselves to study on applications of coffee extracts, or coffee extracts containing arabinogalactan in more detail, the inventors found to have immunomodulating action and achieved the present invention.

The present invention provides an immunomodulating agent comprising coffee extract as active ingredient. Preferably, the coffee extract is immunomodulating agent, which is an extract containing arabinogalactan. The immunomodulating effect of the immunomodulating agent relies on the promotion of the proliferation of an immunocompetent cell such as a macrophage. The immunocompetent cell is preferably any one selected from a macrophage like strain RAW264 or J774.1, a murine splenocyte, a murine peritoneal macrophage and a murine dendrocyte.

The present invention provides a composition comprising the immunomodulating agent. These compositions are available for pharmaceutical composition, a food composition and a cosmetic composition.

A manufacturing step for production of the immunomodulating agent of the present invention comprises a step adding water to green coffee beans, roasted coffee beans and coffee extract residue and heating, a step recovering and concentrating under reduced pressure the heated extract solution, and a step adding ethanol to the heated extraction solution concentrated under reduced pressure and precipitating.

Moreover after a step adding ethanol to the heated extraction solution concentrated under reduced pressure and precipitating, it may be set a step dissolving a precipitation in sodium hydroxide, a step stirring it for 1 to 48 hours at room temperature and next for 1 to 48 hours at 50 degree C. to 70 degree C. a step adjusting pH to 7.0 to 8.0, a step extracting by organic solvent, a step digesting proteins by protease and a step dialyzing by water.

Deionized water, distillated water and mini-Q water is preferably used and distillated water is more preferably used. pH is preferably 7.2 to 7.8, more preferably 7.4 to 7.6, the most preferably 7.95 to 7.55.

Immunomodulating agent of the present invention is characterized by the average molecular weight that is from ten thousands to three millions.

Moreover immunomodulating agent of the present invention is characterized by a ratio of arabinose/galactose of arabinogalactan that is 0.02 to 1.0.

The present invention provides the method to increase production quantity of interleukin-12 (IL-12) of murine splenocyte or murine dendrocyte by adding coffee extract, compared to on coffee extract free.

The present invention provides the method to increase the amount of interleukin-12 (IL-12) in murine blood by administering coffee extract, compared to on non-administration of coffee extract.

The present invention provides the method to enhance proliferation promotion activity by mitogen PMA/Ionomycin of murine splenocyte by ingestion of coffee extract.

### Effects of the Invention

Immunomodulating agent of the present invention possesses cellular immunomodulating activity to enhance the production of IL-12 or IFN-gamma. So it is expected to use for the immunotherapy or prevention of cancer. The active ingredient of immunomodulating agent of the present invention is sugar and/or lactic bacterium that has been used as food, and it is useful as not only pharmaceutical but also food as it is known to be safe.

### Best Mode for Carrying Out the Invention

Hereinafter, embodiments of the present invention will be closely described with referent to the drawings. Immunomodulating agent of the present invention contains coffee extract as the active element. Green coffee beans, residue after extracting coffee, roasted coffee beans are used as materials to get coffee extract, and are not limited to these.

Coffee means coffea plant in the present invention. There are three ancestors of arabica, robusta and liberica, and several breeds based them as cultivated variety of coffea belonging to rubiaceae. Coffea canephona includes and is not limited to these.

In addition, any purification levels such as crude extract (Crude AG), quasi-crude extract (Quasi-crude AG) and highly purified substance etc. maybe used. The bottom line is that it contains an ingredient comprising immunomodulating activity.

Coffee extract is obtained by extraction treatment of a part of coffee plant or residue after extracting coffee. As plant part for extraction, for example, the above part of coffee beans is preferably used but is not limited to these.

Moreover solvent, which is used on extracting, is not particularly limited and may be any one of polar or nonpolar solvent. Concretely polar solvent of water, ethyl acetate and like that is illustrated by an example as the said extract solvent. These solvents may be used solely and may be used by combination of more than two kinds. The extracting solvent is preferably polar one, and more preferably water.

The method to extract is to add distilled water to green coffee beans, roasted coffee beans or coffee extract residue, heating and extracting on rough extracting. And it is to add three to four times (V/V) the amount of ethanol after concentrating under reduced pressure, collecting pellet as rough extract fraction.

For highly purification, crude extract fraction is dissolved in sodium hydrate solution, stirring at room temperature for several hours and successively at 55 to 60 degree C. for several hours, extracting in series with chloroform, ethyl acetate and diethyl ether after adjusting to pH7.0 to 8.0 with acid of sulfuric acid and hydrochloric acid and so on, adding trypsin to water layer, reacted at 40 degree C. for 48 hours and breaking up proteins. Refined arabinogalactan is obtained by dialyzing these to distillated water.

What coffee extract contains arabinogalactan (Coffee AG; Cof-AG) means that the above extract contains arabinogalactan from coffee. Molecular weight of arabinogalactan from coffee is preferably 5000 or more but not exceeding 3 millions, more preferably 10 thousands or more but not exceeding 3 millions, more preferably 20 thousands or more but not exceeding 3 millions. The term molecular weight here is measurement by gel filtration chromatography using HPLC (High Performance Liquid Chromatography).

Ratio of arabinose/galactose of coffee extract is 0.02 to 1.0, more preferably 0.3 to 0.5. This figure can be compartmentalized at any figure within 0.02 to 1.0 because it exerts a certain effect when it is compartmentalized at any part within 0.02 to 1.0.

Compositions containing the above immunomodulating agent are listed for example pharmaceutical composition, food composition and so on.

### (Pharmaceutical Composition)

In the field of medicine, pharmaceutical composition with immunomodulating activity is provided by blending carriers or additives allowed pharmaceutically with coffee extract of the amount, which exerts effectively immunomodulating activity. Such pharmaceutical composition may be either pharmaceutical medicine or quasi drug.

Such pharmaceutical composition may be applied either internally or externally. So such pharmaceutical composition can be used in the form of internal agent, injectable solution of intravenous injection, hypodermic injection, endermic injection, muscle injection and/or intraperitoneal administration and so on, and drug product of transmucosal applicable agent, percutaneous applicable agent and so on.

Formulation of such pharmaceutical composition is properly set according to applied forms, for example solid preparation of tablet, granular form, capsule, powder medicine, powdered drug and so on, liquid medicament of liquid drug, suspension agent and so on, and semisolid preparation of ointment, gel and so on.

Antiviral, anticancer drug, preventive/therapeutic agent for hepatitis, atopic dermatitis and allergy to pollen, antiflatulent and so on are listed as application of such pharmaceutical composition.

### (Food Composition)

In the field of food, food composition with immunomodulating activity is provided by blending coffee extract the effective amount which immunomodulating activity is exerted in vivo into various foods as food materials. The present invention can provide food composition marked the message with for immunomodulation in the field of food. Specified health food, dietary supplement, functional food, food for hospital patients in addition to general food are listed as such food composition.

For example condiment, meat substitute, agricultural processed goods, beverage (cold drink, liquor, carbonated drink, milk beverage, fruit juice drink, tea, coffee, nutritious drink and so on), powder drink (powder juice, powder soup, and so on), condensed juice, confectionary (candy, cookies, biscuit, gum, gummi candy, chocolate and so on), bread, cereal and so on are listed. In case of specified health food, dietary supplement, functional food and so on, any forms of capsules, troche, syrup, granule, powder and so on can be listed.

Although it can be properly determined by experiment, compounding ratio of coffee extract in the food composition is preferable 0.01 mg/L to 5 mg/L for example, and more preferable 0.05 mg/L to 1 mg/L.

Antiflatlent, preventative/therapeutic agent for pollen allergy, food additive, food for atopic dermatitis and so on are listed for example as applications of the food composition.

Immunomodulating agent of the present invention is preferable to contain lactobacillus as active element. Lactobacillus using usually in food processing is used as lactobacillus, and intestinal lactobacillus in the human intestine is more preferable. Lactobacillus gasseri, lactobacillus casei, lactobacillus acidophilus, bifidobacterium longum, bifidbacterium infantis, bifidobacterium bifidum, bifidbacterim breve, bifidobacterium adolescentis, streptococcus faecalis and so on are listed typically. These may be used solely and may be used by combination of more than two kinds.

Immunomodulating agent of the present invention can increase IL-12 productivity of macrophage or IFN-gamma productivity of lymphocyte among intestine epidermal cells by above extract only, combined with others or by using mixture of above lactobacillus.

### (Preparation Example)

Lactobacillus/gasseri JCM1131 was inoculated into 5 ml of MRS medium (trade name “Lactobacilli MRS Broth”, manufactured by Difco Co., Ltd.), lactobacillus culture medium, and was static-cultured at 32 degree C. for 24 hrs. Thus obtained culture solution was centrifuged at 10000×g for 20 min. and bacterial cells were recovered. The cells were suspended in PBS, centrifuged at 10000×g for 20 min and bacterial cells were recovered. After these operations were repeated 3 times, cells were redissolved in distilled water. After this suspension was sterilized by heating at 70 degree C. for 10 min and it was frozen rapidly in dry ice-ethanol. This was freeze-dried and 0.73 g of dried dead cells of Lactobacillus gasseri were obtained.

### EXAMPLE 1

Proliferation Test Using Macrophage like Cell Line RAW264)

Macrophage like cell line RAW264 cell line (available from Riken RCB00535) from mouse were diluted with DMEM medium containing 10% FBS (fetal bovine serum) (after here, the medium is called medium simply) to cell number of 20×10⁵/ml). 50 μl of this was inoculated per well of 96 well tissue culture plate and cultured at 37 degree C. for 2 hrs in 5% CO₂ incubator.

To this, coffee extract obtained by above preparation example was added to medium to become the concentration of 0.0625 μg/ml to 0.5 μg/ml and volume of one well was adjusted to 100 μl. For comparison, arabinogalactan fraction from larch was added to medium at the same concentration. Additionally, substance stimulating immunocyte, that is, LPS (lipopolysaccharide) and conA (concanavalin A) which is known as excellent substance for immunoresponse is added to 20 μg/ml. These were cultured at 37 degree C. for 1 to 4 hrs in 5% CO₂ incubator and proliferated cell volume was monitored. For reagent to conduct proliferation test Premix WST-1 Cell Proliferation Assay System was used and MICROPLATE READER Model 550 manufactured by BIO-RAD Co., Ltd. was used for a measurement equipment.

Results of proliferation test using macrophage like cell line RAW264 is shown in FIG. 1. Significant proliferation promotion activity was recognized compared to control when arabinogalactan from coffee was added in murine macrophage like cell line. In addition, proliferation promotion activity significant to control or slightly significant to control was recognized when 3 kinds of extracts, extract of coffee extract residue (Crude AG from residue; CrudeR-AG), quasi crude extract (Quasi-crude AG from green coffee beans; Q. CrudeB-AG) and extract of green coffee beans (Crude AG from green coffee beans; CrudeB-AG) were added.

### EXAMPLE 2

### (Proliferation Test Using Murine Splenocytes)

Splenocytes were prepared from mouse and proliferation promotion activity was investigated as example 1. Cells were diluted to cell number of 100×10⁵/ml with RPMI1640 medium containing 10% FBS (fetal bovine serum) (hereinafter the medium is called medium simply). 50 μl of this was inoculated per cell of 96 well tissue culture plate and was cultured at 37 degree C. for 2 hrs in 5% CO₂ incubator. To this, coffee extract obtained in above preparation example was added to medium to the concentration of 0.125 μg/ml to 0.5 μg/ml and total volume of one well was adjusted to 100 μl. For comparison, arabinogalactan fraction from larch was added to medium at the same concentration. These were cultured at 37 degree C. for 1 to 4 hrs and growth quantity was monitored. As reagent for proliferation test, Premix WST-1 Cell Proliferation Assay System and MICROPLATE READER Model 550 manufactured by BIO-RAD Co., Ltd. was used for a measurement equipment.

Results of proliferation test using murine splenocytes are shown in FIG. 2-1 to FIG. 2-3. FIG. 2-1 to FIG. 2-3 shows growth activity of splenocytes of respective arabinogalactan containing fraction (respective crude extracts of coffee extract residue, green coffee beans extract and quasi-crude extract). In inbred splenocytes, when arabinogalactan from coffee was added, significant increase of growth activity was observed compared to control. In addition, in inbred balb/c mouse significant difference of growth activity between arabinogalactan from larch and arabinogalactan from coffee was recognized. In addition, the same or higher growth activity as highly purified pure substance of coffee extract was recognized about crude extract.

When taken together, coffee extract had higher proliferation promotion activity than arabinogalactan from larch. In addition, significant proliferation promotion activity was observed about any of coffee extract residue extract, quasi-crude extract and green bean extract.

### EXAMPLE 3

### (Proliferation Test Using Murine Peritoneal Macrophage)

Macrophages were prepared from murine abdominal cavity and were diluted to macrophage cell number of 10×10⁵/ml with Hanks' Balanced Salt Solutions. 50 ml of this was inoculated per one well of 96 well tissue culture plate and was cultured at 37 degree C. for 2 hrs in 5% CO₂ incubator. After culture, nonadherent cells were removed by washing with Hanks' Balanced Salt Solutions and 50 ml/well of RPMI1640 medium containing 10% FBS (fetal bovine serum) (hereinafter called medium simply) was added. To this, coffee extract obtained by above preparation example was added to medium to the concentration of 0.125 μg/ml to 0.5 μg/ml. For comparison, arabinogalactan fraction from larch was added to the same concentration and volume per well is adjusted to 100 μl. These were cultured at 37 degree C. for 2 to 4 hrs and proliferated cell volume was monitored. As reagent for proliferation test, Premix WST-1 Cell Proliferation Assay System of Takara Bio Co., Ltd. and MICROPLATE READER Model 550 manufactured by BIO-RAD co. Ltd. was used for a measurement equipment.

Results of proliferation test using murine peritoneal macrophage are shown in FIG. 3-1 to FIG. 3-3. Compared to arabinogalactan from larch, coffee extract had higher proliferation promotion activity. It was observed that proliferation activity increased significantly compared to control when arabinogalactan from coffee was added in Murine peritoneal macrophage of both lines of inbred and closed colony. In addition, when growth activity effect of arabinogalactan from larch and that of arabinogalactan from coffee were compared, there was significant difference. That is, 1.4-fold activation of macrophage growth was observed. Especially, effect of crude extract surpassed purified one compared to splenocytes significantly.

### EXAMPLE 4

### (ELISA Test Using Murine Splenocytes)

Splenocytes were prepared and expression of cytokine was investigated. Cell suspension was diluted to 60×10⁵/ml with RPMI1640 medium containing 10% FBS (fetal bovine serum) (after this, it is called medium simply).

500 μl/well of this was inoculated in 24 well culture plates and cultured in 5% CO₂ incubator at 37 decree C. To this, coffee extract obtained as preparation example above was added to the concentration of 0.25 μg/ml and total volume is made to 1 ml per one well.

For comparison, arabinogalactan fraction from larch was added to medium at the same concentration. After cultivation in 5% CO₂ incubator at 37 degree C. for 20 to 37 hrs, supernatant was recovered. Detection (of expression) of IL-12 quantity and IFN-gamma quantity was done by Immuno assay Kit IL12p40 and Immunoassay Kit IFN-gamma (BIOSOURCE) respectively. MICROPLATE READER Model 550 manufactured by BIO-RAD Co., Ltd. was used as measurement equipment.

### (ELISA Test Using Culture Supernatant of Murine Dendrocytes

Dendrocytes from mouse were prepared and expression of cytokine was investigated. Bone-marrow cells were collected from mouse lower limb using a syringe and diluted to cell number of 4×10⁶/ml with RPMI1640 medium containing 4 ng/ml of IL-4 (Wako chemicals Co., Ltd.) and 10 ng/ml of GM-CSF (Wako chemicals Co., Ltd.) and 10% FBS (fetal bovine serum) (this medium is called medium simply after this). 250 μl/well of this was inoculated and cultured in 5% CO₂ incubator at 37 degree C. for 1 week. In addition, cells were washed and removed on 2^{nd} and 4^{th} day in 1-week culture period. Coffee extract obtained as above preparation example was added to this at the concentration of 0.25 μg/ml to 500 μl total per one well. For comparison, arabinogalactan fraction from larch was added at the same concentration to the medium.

Supernatant was recovered after culturing these cells at 37 degree C. for 20 hrs in 5% CO2 (CO₂) incubator. Detection (of expression) of IL-12 quantity was done using Immunoassay Kit Mouse IL-12p40 (BIOSOURCE), detection (of expression) of IFN-gamma (BIOSOURCE) using Immunoassay Kit Mouse IFN-gamma (BIOSOURCE). MICROPLATE READER Model 550 manufactured by BIO-RAD Co., Ltd. was used as measurement equipment.

Results of ELISA test using culture supernatant of murine splenocyte and ELISA test using culture supernatant of murine dendrocytes are shown in FIG. 4-1 to FIG. 4-3, FIG. 9-1 and FIG. 9-2. It was observed that the concentration of IL-12 of splenocyte supernatant was slightly significant to control and concentration of IL-12 and IFN-gamma of dendrocyte supernatant increased significantly to control (FIG. 4-1 to FIG. 4-3) when splenocytes and dendrocytes isolated from inbred balb/c mouse was added with 0.25 μg/ml arabinogalactan from coffee and was cultured for 20 hrs.

In addition, 0.25 μg/ml of arabinogalactan from coffee was added to splenocytes isolated from the same mouse and the cells are cultured for 37 hrs Then, concentrations of both IL-12 and IFN-gamma of supernatant had a trend to increase and especially significant difference was recognized in IFN-gamma production in coffee extract residue (refer to FIG. 9-1 and FIG. 9-2).

### (ELISA Test Using Macrophage)

Macrophage (Peritoneal macrophage) was prepared from murine abdominal cavity and expression of cytokine was investigated. Cells were diluted to 12×10⁵/ml with Hanks' solution. 500 μl of this was inoculated in a well of 24 well tissue culture plate and cultured at 37 degree C. for 2 hrs in 5% CO₂ incubator. After culture, nonadherent cells were removed by washing with Hanks' solution and 500 μl per well of RPM11640 media containing 10% FBS (fetal bovine serum) (after this, the medium is called medium simply) was added. To this, coffee extract was added to medium at the concentration of 0.25 to 250 μg/ml and total volume of 1 well was adjusted to 1 ml.

These were cultured at 37 degree C. for 20 to 48 hrs in 5% CO₂ incubator and supernatant was recovered. Detection (of expression) of IL-12 and TNF-α quantity was conducted using Immuno assay Kit IL-12p40 and Immuno assay Kit TNF-α (BIOSOURCE) respectively. MICROPLATE READER Model 550 manufactured by BIO-RAD Co., Ltd. is used as measurement equipment.

Results of ELISA test using murine macrophage are shown in FIG. 9-3 and FIG. 9-4. Macrophage isolated from inbred balb/c mouse was added with 0.25 to 250 μg/ml of arabinogalactan from coffee and was cultured for 20 to 48 hrs. It is observed that TNF-α concentration of supernatant was concurrently increased according to concentration to control and increasing trend about IL-12 was recognized especially in coffee extract residue.

In addition, J774.1 cell line which is macrophage like cell line from mouse (obtainable from Riken RCB0434) was diluted to 2.4×10⁵/ml of cell number with RPMI1640 medium containing 10% FBS (fetal bovine serum) (after this the medium is called medium simply). 500 ml of this was inoculated per well of 24 well tissue culture plate and was cultured at 37 degree C. for 1 hr in 5% CO₂ incubator. To this, coffee extract obtained in above preparation example was added to the concentration of 25 to 5000 μg/ml and total volume of one well is adjusted to 1 ml. These were cultured at 37 degree C. for 20 hrs in 5% CO₂ incubator and supernatant was recovered. Detection (of expression) of TNF-α quantity was conducted using Immuno assay Kit TNF-α (BIOSOURCE). MICROPLATE READER Model 550 manufactured by BIO-RAD co., Ltd. is used as measurement equipment.

Results of ELISA test using J774.1 cell line are shown in FIG. 9-5. J774.1 cell line was added with 25 to 5000 μg/ml of arabinogalactan from coffee and was cultured for 20 hrs. It was observed that concentration of TNF-α of supernatant was concurrently increased compared to control.

(Proliferation Test Using Murine Splenocytes after Administration of Arabinogalactan)

Inbred balb/c mouse of 9 week male were used for arabinogalactan administration test and conducted for 1 week as following administration quantity and number (n).

Purified arabinogalactan from coffee 2.5 mg/day; n=5 Purified arabinogalactan from larch 2.5 mg/day; n=5 Crude extract of coffee extract residue 2.5 mg/day; n=5 Water (control); n=6

In addition, each sample was dissolved in water and administration form was to ingest it freely. Splenocytes from mouse were prepared and proliferation promotion activity for mitogen PMA/Ionomycin was investigated. These were diluted to cell number of 20×10⁵/ml with RPMI1640 medium containing 10% FBS (fetal bovine serum) (after this the medium called medium simply). 50 μl of this was inoculated in a well of 96 well tissue culture plate. To this, PMA (SIGMA) was added to final concentration of 50 ng/ml and Ionomycin (SIGMA) to 1 ng/ml and total volume was adjusted to 100 μl per well.

These are cultured at 37 degree C. for 24 hrs in 5% CO₂ incubator and proliferated cell volume was monitored. Premix WST-1 Cell Proliferation Assay System manufactured by Takara Bio Co.; Ltd. for a reagent conducting proliferation test and MICROPLATE READER Model 550 manufactured by BIO-RAD Co., Ltd. for a measurement equipment.

(Proliferation Test Using Murine Splenocytes after Administration of Arabinogalactan)

Results of proliferation test for PMA/Ionomycin using administrated murine splenocytes are shown in FIG. 5-1. Growth activity increased with slight significance (p<0.09) when murine splenocytes administrated with arabinogalactan from coffee were added with mitogen and cultured for 24 hrs. In addition, significant increase of proliferation activity was acknowledged in murine splenocytes administered with coffee extract residue compared to control.

(ELISA Test Using Murine Blood Serum after Administration of Arabinogalactan)

After the end of arabinogalactan administration test above, murine serum was recovered and expression of cytokine was investigated. Confirmation of IL-12 quantity was done using Immunoassay Kit IL-12+p40 (BIOSOURCE). MICROPLATE READER Model 550 manufactured by BIO-RAD Co., Ltd. as measurement apparatus. Results are shown in FIG. 5-2. 2.5 mg of purified arabinogalactan and crude extract from extract residue per day for a week to inbred balb/c mouse and cytokine concentration in blood was observed to be enhanced significantly against control.

### (Preparation of Arabinogalactan)

100 g of coffee green beans or coffee extract residue was added with 1500 to 2000 ml of distilled water and was extracted at 121 degree C. for 2 hrs. Thus obtained extract was centrifuged at 10000 rpm for 20 minutes. The supernatant was recovered, concentrated by rotary evaporator under reduced pressure, added with 3 to 4 times volume of ethanol and the precipitate was recovered. A preparation flow of arabinogalactan from coffee green beans is shown in FIG. 6(A) and coffee extract residue in FIG. 6(B).

For further purification, 2.5 g of crude purified substance was dissolved in 100 ml of 0.2M sodium hydrate, stirred at room temperature (25 degree C.), at 55 to 60 degree C. for 3 hrs, adjusted to pH7.5 with sulfuric acid, extracted sequentially with chloroform, ethyl acetate, diethyl ether, added with trypsin to aqueous phase, reacted at 40 degree C. for 48 hrs and protein was removed by degradation. By dialyzing this to distilled water, 1.4 g of purified arabinogalactan was obtained. The preparation flow is shown in FIG. 7.

### (Measurement of Average Molecular Weight of Purified Arabinogalactan)

Average molecular weight was measured using pullulan (Showa denko Ltd. Produced) as standard by gel permeation chromatography using HPLC (column: TSK-GEL G6000PW φ 7.5 mm×300 mm, guard column: TSK-GUARD COLUMN PWH 7.5×75 mm, transfer phase: 0.1M NaCl in 0.1M phosphate buffer (pH6.6), detector: RI, detection temperature: 45 degree C., flow rate: 0.2 ml/min).

### EXAMPLE 5

Example 5 shows results investigated suppression effect of total IgE antibody production in blood by administration of arabinogalactan from coffee. Suppression'effect of total IgE antibody production in blood was confirmed when mouse fed with arabinogalactan from coffee or arabinogalactan from larch was administered with egg albumin (OVA, SIGMA) and IgE antibody production was induced.

Specifically, induction of IgE antibody production by OVA was done using female inbred balb/c mouse under test section and number (n) of following (A) to (C).
(A) Water (control); n=6
(B) Purified arabinogalactan from coffee 2.5 mg/day; n=5
(C) Purified arabinogalactan from larch 2.5 mg/days; n=5

Sensitization was conducted as follows. As first sensitization, OVA solution was prepared in which 10 μg of OVA and 2 mg of hydroxyl aluminum gel (SIGMA) as adjuvant was suspended in 0.3 ml of phosphate buffered saline (PBS) solution and was administered intraperitoneally to 6-week-old mouse at the first day and 4^{th} day from the start of sensitization. As 2^{nd} sensitization, OVA was dissolved in PBS to 25 mg/ml and mouse nose was exposed to this antigen solution for about 3 seconds. This procedure was repeated 3 times for one experiment. Operation of 2^{nd} sensitization was conducted twice a day morning and afternoon 10 days after the first sensitization everyday for 10 days.

Administration of arabinogalactan was started 1 week before first sensitization. In addition, each sample was dissolved in water and ingested freely.

Blood was collected after 20 days from the start of 1^{st} sensitization and total IgE concentration was investigated by collecting blood serum. Total concentration of IgE antibody in blood was measured by Morinaga mouse IgE Kit (Morinaga Institute of biological Science, Inc.

As shown in FIG. 10, decreasing trend of total IgE quantity was confirmed in the experimental section of 2.5 mg/day of purified arabinogalactan from coffee compared to that of control and purified arabinogalactan from larch.

### EXAMPLE 6

### (Proliferation Test for Enteric Bacteria)

Assimilating activity of arabinogalactan from coffee (Cof-AG) by bacteria which constitues enteric bacteria flora is shown as follows including results of comparison with other sugars.

GAM bouillon liquid medium used for preculture and for test medium, Pepton-Yeast-Fildes solution (PYF) liquid medium was used after addition of test sugar and autoclaved. PYF medium above comprises composition of table 1 below. And Fildes solution is prepared as table 2 below.

TABLE 1

| Composition | Weight | |
|---|---|---|
| Trypticase peptone | 10.0 | g |
| Yeast extract | 10.0 | g |
| Fides solution | 40.0 | mL |
| Salt solution | 40.0 | mL |
| CaCl2 | 0.1 | g |
| MgSO4•7H20 | 0.2 | g |
| K2HPO4 | 0.5 | g |
| KH2PO4 | 0.5 | g |
| NaHCO3 | 5.0 | g |
| NaCl | 1.0 | g |
| NilliQ | 500.0 | mL |
| L-cystein-HCL | 0.5 | g |
| Carbon source | | |
| Glucose | 5.0 | g |
| Or | | |
| Arabinogalactan from coffee(Cof-AG) | 20.0 | g |
| Or | | |
| Arabinogalactan from larch(L-AG) | 20.0 | g |
| MilliQ | 920.0 | mL |
| (pH7.2) | | |

TABLE 2

| | |
|---|---|
| Normal saline (0.85%, NaCl) | 150 mL |
| Hydrochloride (arsenic-free) | 6 mL |
| Horse defibered blood | 50 mL |
| Pepsin (1:10000) | 5 g |
| 20% NaOH solution | 12 mL |

Ingredients above was mixed, maintained at 55 degree C. in water bath overnight and digested. After 12 ml of 20% NaOH solution was added to this mixture, pH of it was adjusted to 7.6 and was sterilized through a filter.

### (Examination Method and Determination of Results)

Fresh bacteria cultured in GAM bouillon was inoculated in PYF medium added with supply sugar for each strain to 10⁷ to 10⁸ respectively and anaerobic cultured at 37 degree for 72 hrs. Growth of bacteria number by pH decrease of medium was determined 72 hrs after inoculation. Determination basis is as follows: (pH of sample)—(pH of sugar depleted medium)=[pH]; [pH]<0.5 is set to (−); 0.5<=[pH]<1.0 to (±), 1.0<=[pH]<1.5 to (+) and 1.5<=[pH] to (++).

In addition, about examination methods and determination methods, refer to the literature ┌Suzuki et al, Utilization by Intestinal Bacteria and Digestibility of Arabino-oligosaccharides In Vitro (J. Japan. Soc. Hort. Sci. 73(6): 574-579, 2005)┘ As for carbon source, as shown in table 1, glucose (control), arabinogalactan from coffee and arabinogalactan from larch were used. In addition, strains provided are shown in the following table 3. Experimental results are shown in the following table 4 and table 5.

TABLE 3

Bifidobacterium adolescentis JCM1275
Bifidobacterium angulatum JCM7086
Bifidobacterium bifidum NBRC100015
Bifidobacterium breve JCM1192
Bifidobacterium breve JCM7016
Bifidobacterium catenulatum JCM1194
Bifidobacterium dentium JCM1195
Bifidobacterium gallicum JCM8224
Bifidobacterium infantis JCM1222
Bifidobacterium longum JCM1217
Bifidobacterium longum JCM7052
Bifidobacterium longum JCM7053
Bifidobacterium longum JCM7054
Bifidobacterium longum JCM7055
Bifidobacterium longum JCM7056
Bifidobacterium longum JCM11340
Bifidobacterium longum JCM11341
Bifidobacterium longum JCM11343
Bifidobacterium pseudocatenulatum JCM1200
Enterococcus faecalis NBRC100480
Lactobacillus acidophilus NBRC13951
Lactobacillus casei JCM1134
Lactobacillus gasseri JCM1131
Lactobacillus paracasei JCM8130
Lactobacillus plantarum NBRC3070
Lactobacillus rhamnosus NBRC3425
Lactobacillus salivarius JCM1231
Lactococcus lactis NBRC100933
Clostridium butyricum NBRC3315
Clostridium kainantoi NBRC3353
Clostridium paraputirificum JCM1293
Clostridium ramosum JCM1298
Clostridium sporogenes NBRC13950
Escherichia coli NBRC3301

TABLE 4

| Strains | glucose | Cof-AG | L-AG |
|---|---|---|---|
| Bifidobacterium longum JCM1217 | ++ | ++ | ++ |
| Bifidobacterium longum JCM7052 | ++ | ++ | ++ |
| Bifidobacterium longum JCM7053 | ++ | ++ | ++ |
| Bifidobacterium longum JCM7054 | ++ | ++ | ++ |
| Bifidobacterium longum JCM7055 | ++ | ++ | ++ |
| Bifidobacterium longum JCM7056 | ++ | ++ | ++ |
| Bifidobacterium pseudocatenulatum JCM1200 | ++ | ++ | ± |

TABLE 5

| Strains | glucose | Cof-AG | L-AG |
|---|---|---|---|
| Clostridium butyricum NBRC3315 | + | − | − |
| Clostridium kainantoi NBRC3353 | + | − | − |
| Clostridium paraputirificum JCM1293 | + | − | − |
| Clostridium ramosum JCM1298 | + | − | − |
| Clostridium sporogenes NBRC13950 | ++ | − | − |
| Escherichia coli NBRC3301 | + | − | − |

As clear from above table 4, arabinogalactan from coffee was well assimilated by Bifidobacterium, enteric useful bacterium, equivalent to arabinogalactan from larch by Bifidobacterium longum and better than arabinogalactan from larch by Bifidobacterium pseudocatenulatum.

And from results of table 5, arabinogalactan from coffee was hardly assimilated by Clostridium genus or Escherichia coli that is classified as enteric harmful bacteria.

Here, Bifidobacterium genus is known as representative bacterial species of enteric useful bacteria in human. From above explanation, it may be understood that prebiotics effect such as improvement of enteric environment by eating arabinogalactan from coffee was expected.

### INDUSTRIAL APPLICABILITY

Immunomodulating agent of the present invention comprises cellular immunomodulating function because of promotion of macrophage growth. So, utilization as cancer immunotherapy, cancer prevention, preventive/treatment drug for viral disease is expected. And it can be used for health food or effective utilization of coffee extract residue.

### BRIEF DESCRIPTION OF THE DRAWINGS

FIG. 1 is a figure of investigation of proliferation promotion activity of coffee extract using macrophage like cell line RAW264.

FIG. 2-1 is a figure of investigation of proliferation promotion activity (balb/c) of coffee extract using murine splenocytes.

FIG. 2-2 is a figure of investigation of proliferation promotion activity (C57BL/6) of coffee extract using murine splenocytes.

FIG. 2-3 is a figure of investigation of proliferation promotion activity (ICR) of coffee extract using murine splenocytes.

FIG. 3-1 is a figure of investigation of proliferation promotion activity (balb/c) of coffee extract using murine peritoneal macrophage.

FIG. 3-2 is a figure of investigation of proliferation promotion activity (C57BL/6) of coffee extract using murine peritoneal macrophage.

FIG. 3-3 is a figure of investigation of growth promoting activity (ICR) using murine peritoneal macrophage.

FIG. 4-1 is a figure of investigation of increase of IL-12 concentration by adding arabinogalactan from coffee to splenocytes isolated from inbred balb/c mouse.

FIG. 4-2 shows results of investigation of increase of IL-12 concentration by adding arabinogalactan from coffee to dendrocytes isolated from inbred balb/c mouse.

FIG. 4-3 shows investigation of increase of IFN-gamma concentration by adding arabinogalactan from coffee to dendrocyte isolated from inbred balb/c mouse.

FIG. 5-1 shows a result of proliferation test for PMA/Ionomycin using the splenocytes administrating purified arabinogalactan from coffee to inbred balb/c mouse for a week.

FIG. 5-2 is a figure investigating increase of cytokine concentration in blood administrating purified arabinogalactan from coffee to inbred balb/c mouse for a week.

FIG. 6 shows preparation method for arabinogalactan from coffee green beans and coffee extraction residue.

FIG. 7 shows preparation method for arabinogalactan from coffee beans.

FIG. 8 shows average molecular weight of purified arabinogalactan.

FIG. 9-1 shows results of ELISA test (IL-12 production) using murine splenocytes.

FIG. 9-2 shows results of ELISA test (IFN-gamma production) using murine splenocytes.

FIG. 9-3 shows results of ELISA test (IL-12 production) using murine macrophage.

FIG. 9-4 shows results of ELISA test (TNF-α production) using murine macrophage.

FIG. 9-5 shows results of ELISA test (TNF-α production) using J774.1 cells.

FIG. 10 is a figure showing results investigating suppression effect of total IgE antibody production in blood by administration of coffee from arabinogalactan.

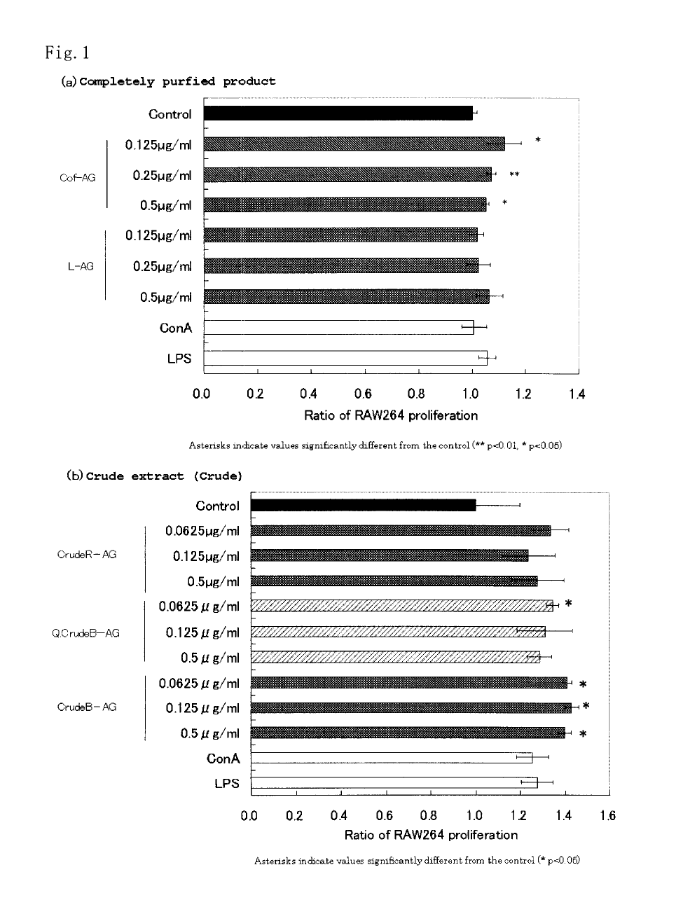

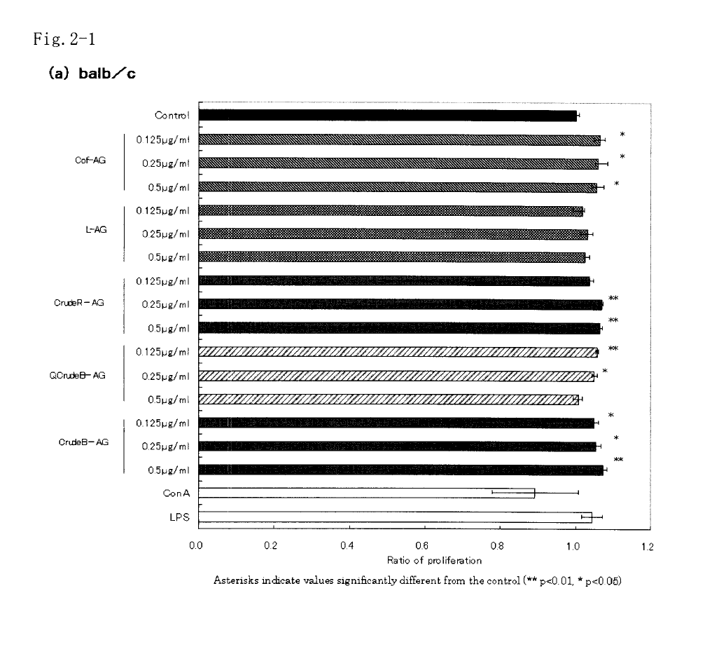

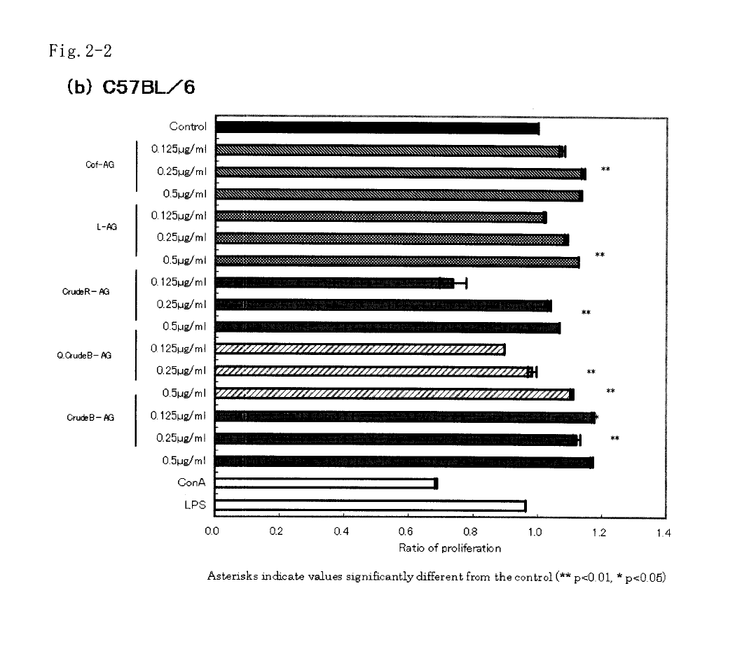

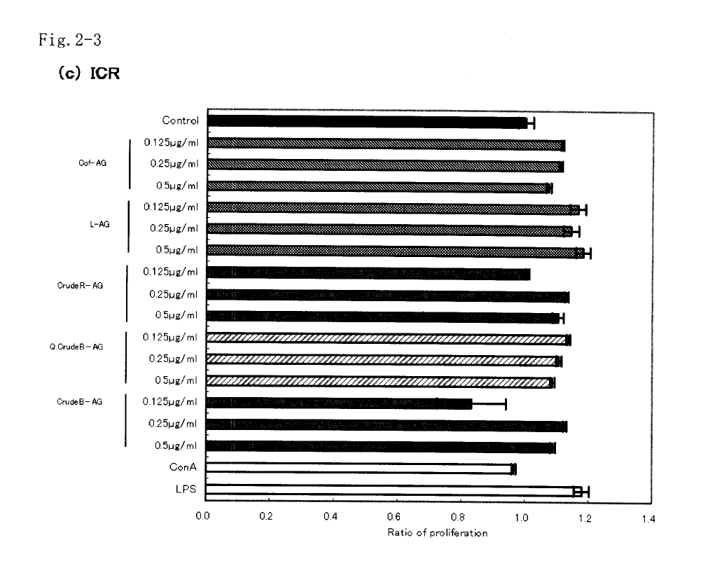

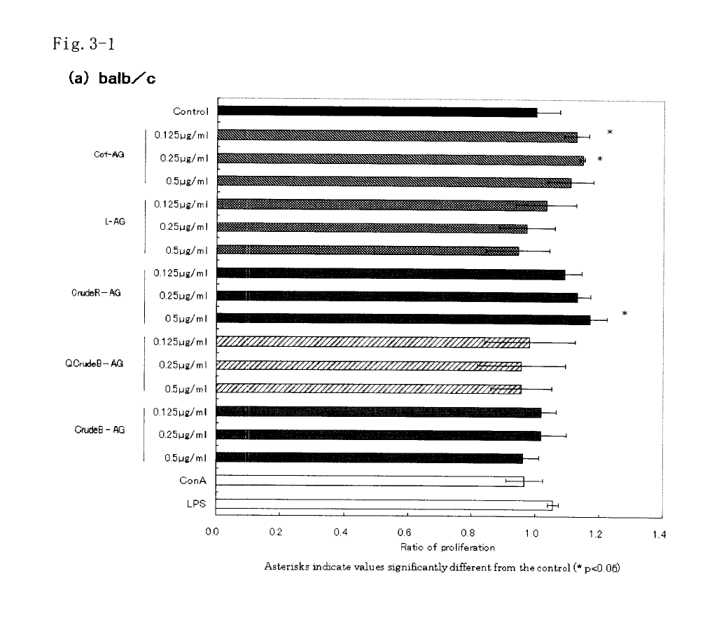

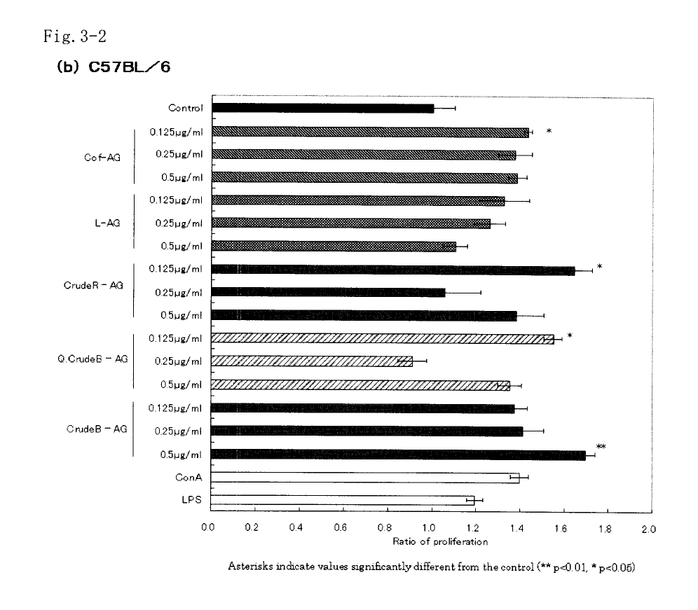

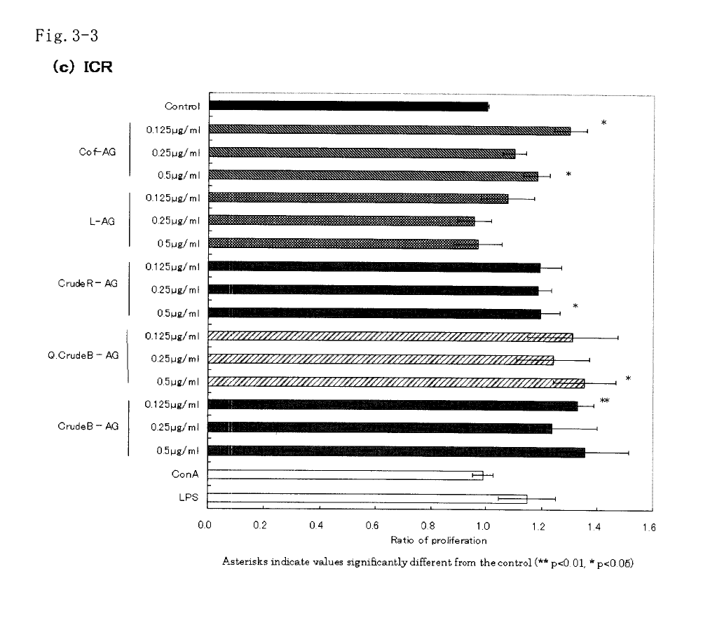

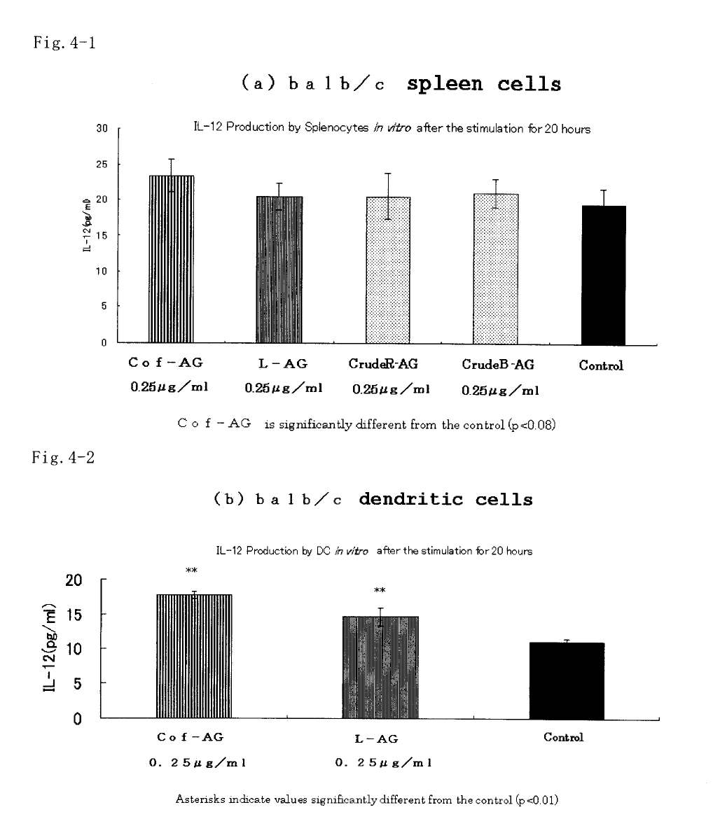

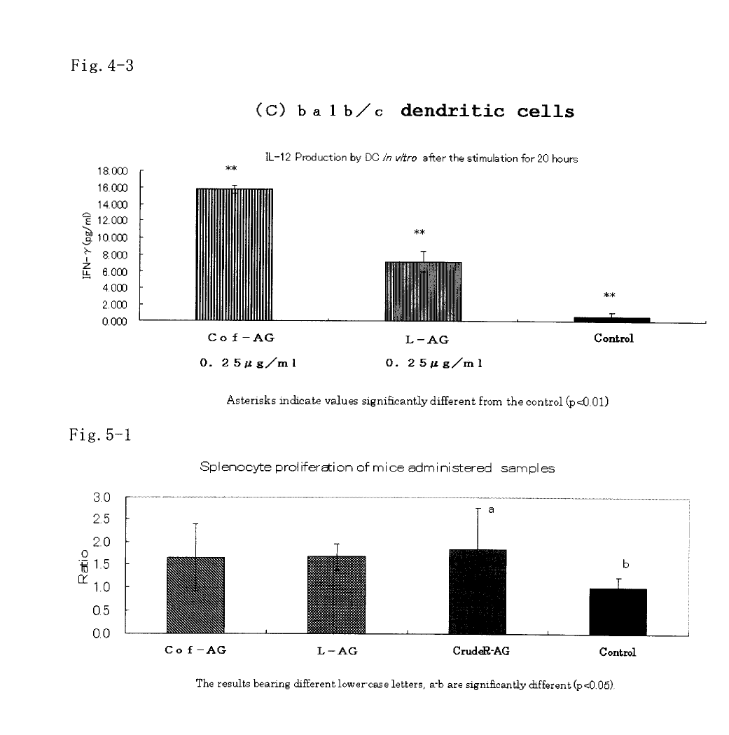

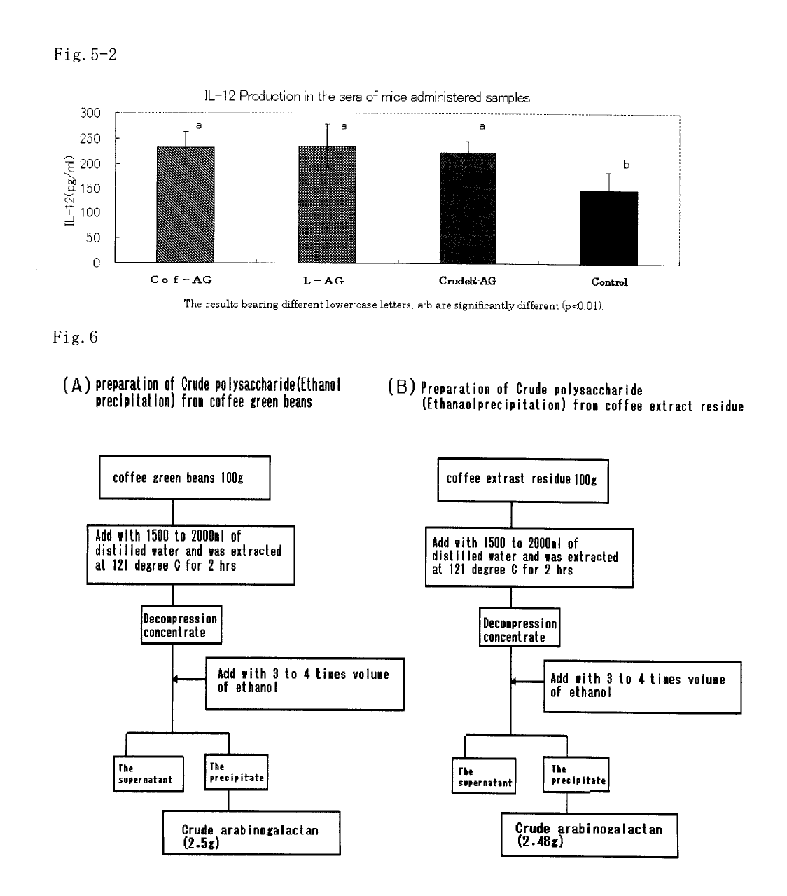

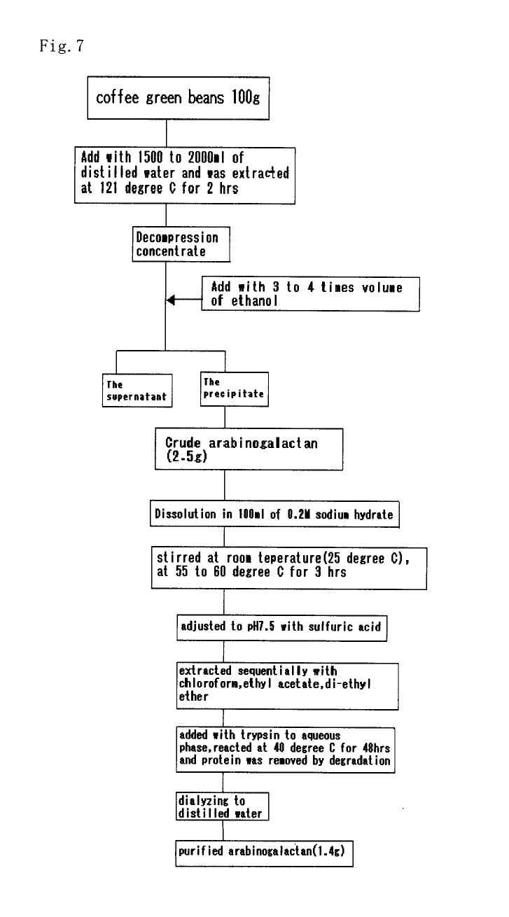

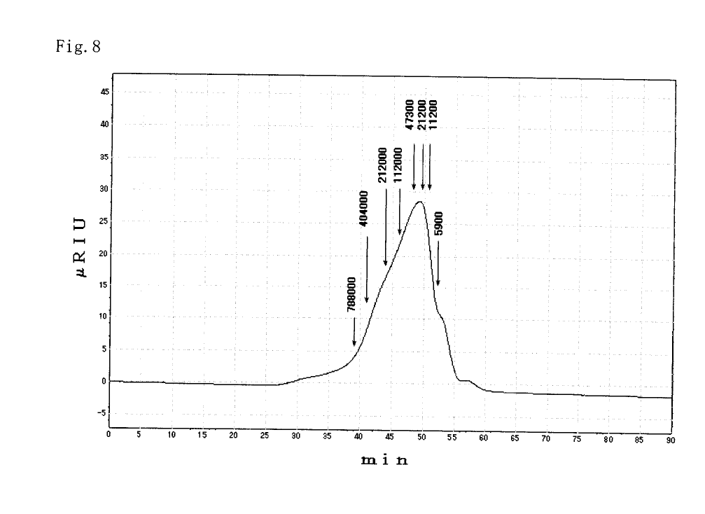

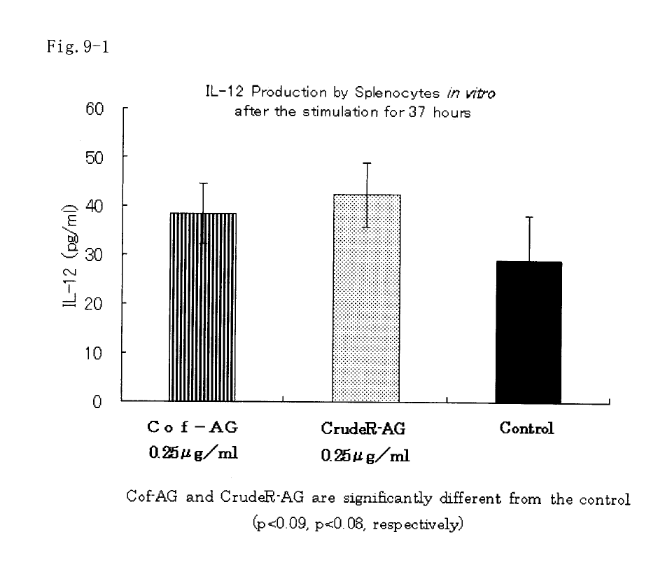

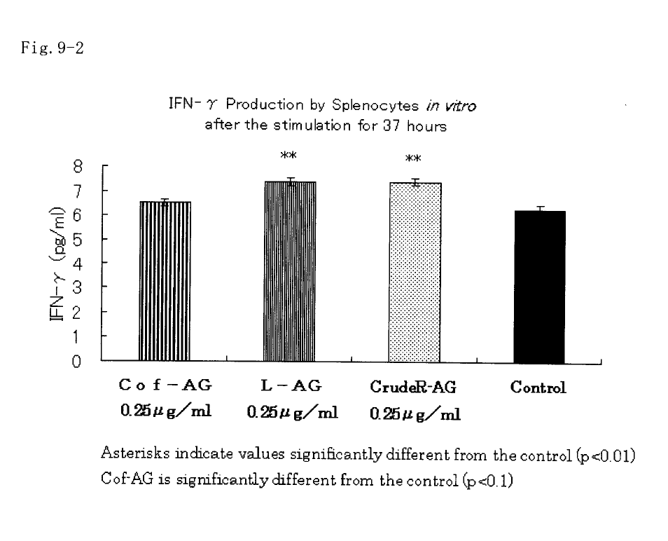

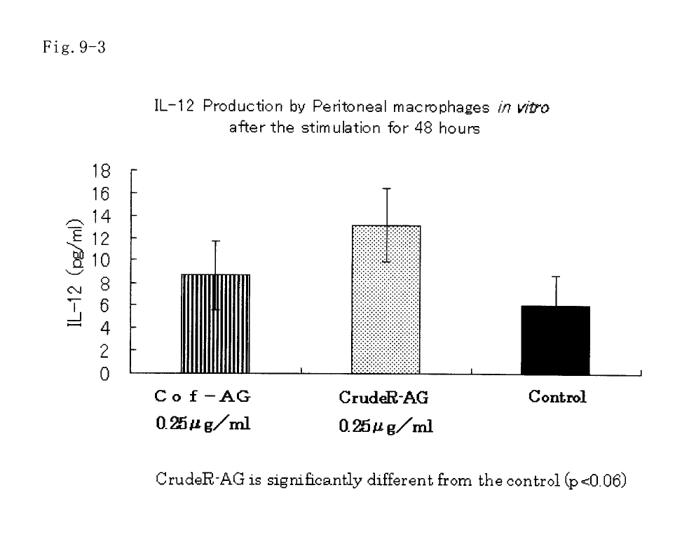

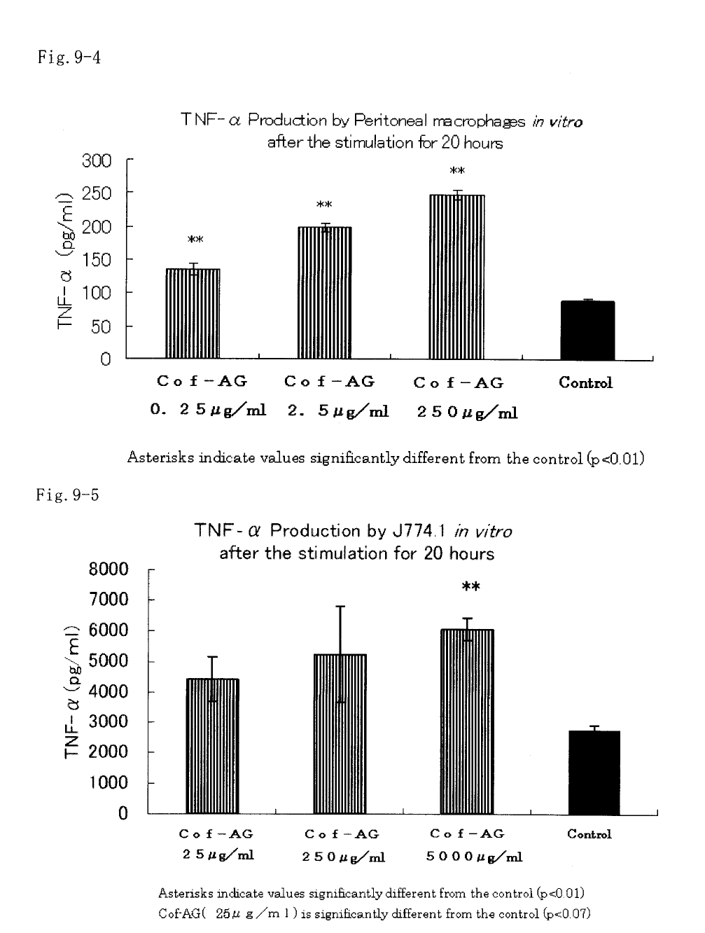

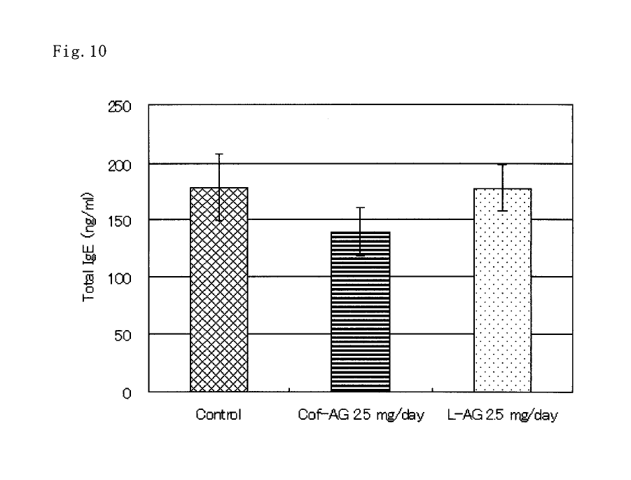

1-47. (canceled)

48. A method of using arabinogalactan obtained from coffee extract as an immunomodulating agent, comprising:
obtaining a composition which includes arabinogalactan (AG) from coffee extract as an active ingredient; and
administering the composition, thereby promoting an immunomodulating activity.

49. The immunomodulating agent usage method of claim 48 wherein the immunomodulating activity comprises proliferation of immunocompetent cells, namely, macrophage.

50. The immunomodulating agent usage method of claim 48 wherein the immunomodulating activity comprises proliferation of any of: macrophage in cell line RAW264, macrophage in J774.1, murine splenocyte, murine peritoneal macrophage and murine dendrocyte (DC).

51. The immunomodulating agent usage method of claim 48 wherein the average molecular weight of arabinogalactan from coffee extract, in the composition, is in the range from 212,000 to 3,000,000.

52. The immunomodulating agent usage method of claim 48, wherein administering the coffee extract arabinogalactan composition comprises at least one of the following: intravenous injection, hypodermic injection, endermic injection, muscle injection, intraperitoneal administration, administration of a drug product containing a transmucosal applicable agent, administration of a drug product containing a percutaneous applicable agent.

53. The immunomodulating agent usage method of claim 48, wherein administering the coffee extract arabinogalactan composition comprises administering at least one of the following: a pharmaceutical composition in tablet form, a pharmaceutical composition in granular form, a pharmaceutical composition in capsule form, a powder medicine, a powdered drug, a liquid medicament of liquid drug, a semisolid preparation of ointment, a gel.

54. The immunomodulating agent usage method of claim 48, further comprising confirming the immunomodulating activity by measuring at least one of the following: splenocyte growth, macrophage growth, dendrocyte growth, an increase in interleukin 12 quantity.

55. The immunomodulating agent usage method of claim 48, further comprising confirming the immunomodulating activity by measuring at least one of the following: immunocompetent cell growth, an increase in interleukin quantity.

56. The immunomodulating agent usage method of claim 48 wherein the average molecular weight of arabinogalactan from coffee extract, in the composition, is in the range from 404,000 to 3,000,000.

57. The immunomodulating agent usage method of claim 48 wherein the average molecular weight of arabinogalactan from coffee extract, in the composition, is in the range from 788,000 to 3,000,000.

58. An immunomodulating agent production method comprising:
adding water to at least one of the following and heating: coffee green beans, coffee roasted beans, coffee extract residue;
recovering a heated extract solution and condensing under decreased pressure;
adding ethanol to condensed extract and precipitating; and
obtaining refined arabinogalactan from the precipitate.

59. The immunomodulating agent production method of claim 58 wherein obtaining refined arabinogalactan comprises:
redissolving the precipitate in sodium hydride solution;
stirring the solution at room temperature for 1 to 48 hrs and successively at 50 degree C. to 70 degree C. for 1 to 48 hrs;
adjusting pH to 7.0 to 8.0;
digesting protein by proteinase; and
dialyzing in water.

60. The immunomodulating agent production method described in claim 58 wherein the average molecular weight of refined arabinogalactan is in the range from 10,000 to 3,000,000.

61. The immunomodulating agent production method of claim 58 wherein obtaining refined arabinogalactan comprises:
redissolving the precipitate in sodium hydrate solution;
stirring the solution at room temperature for least 2 hours and successively at 55 degree C. to 60 degree C. for at least 2 hours;
adjusting pH to 7.0 to 8.0;
breaking up protein by adding trypsin to water layer and reacting at 40 degree C. for at least 48 hours; and
dialyzing to distillated water.

62. The immunomodulating agent production method of claim 58 wherein obtaining refined arabinogalactan comprises:
redissolving the precipitate;
stirring at room temperature and successively at 50 degree C. to 70 degree C.;
adjusting pH to 7.0 to 8.0;
extracting in series with chloroform, ethyl acetate and diethyl ether;
breaking up protein; and
dialyzing to distillated water.

63. The immunomodulating agent production method of claim 58 wherein the average molecular weight of refined arabinogalactan is in the range from 47,300 to 3,000,000.

64. The immunomodulating agent production method of claim 58 wherein the average molecular weight of refined arabinogalactan is in the range from 112,000 to 3,000,000.

65. The immunomodulating agent production method of claim 58 wherein the average molecular weight of refined arabinogalactan is in the range from 212,000 to 3,000,000.

66. The immunomodulating agent production method of claim 58 wherein the average molecular weight of refined arabinogalactan is in the range from 404,000 to 3,000,000.

67. The immunomodulating agent production method of claim 58 wherein the average molecular weight of refined arabinogalactan is in the range from 788,000 to 3,000,000.
